(19) United States
(12) Patent Application Publication
(10) Pub. No.: US 2010/0261175 A1
(43) Pub. Date: Oct. 14, 2010

(54) USE OF SHORT OLIGONUCLEOTIDES FOR REAGENT REDUNDANCY EXPERIMENTS IN RNA FUNCTIONAL ANALYSIS

(75) Inventors: Soeren Vestergaard Rasmussen, Nivaa (DK); Soeren Moeller, Holte (DK); Torsten Bryld, Koebenhavn (DK)

Correspondence Address: CLARK & ELBING LLP, 101 FEDERAL STREET, BOSTON, MA, 02110, US

(73) Assignee: Exiqon A/S, Vedbaek (DK)

(21) Appl. No.: 12/664,360

(22) PCT Filed: Jun. 12, 2008
(86) PCT No.: PCT/DK08/00215

§ 371 Date: May 7, 2010

Related U.S. Application Data
(60) Provisional application No. 60/936,093, filed on Jun. 18, 2007.

(30) Foreign Application Priority Data
Jun. 15, 2007 (DK) PA 2007 00869

(51) Int. Cl. C12Q 1/68

(52) U.S. Cl. 435/6

(57) ABSTRACT

The present invention relates to functional analysis of miRNAs or other short non-coding RNAs involving the use of two or more sequence distinct miRNAs antagonising oligomeric compounds, which enables the reagent redundancy experiments to reduce the risk of reporting false positive effects of miRNA/ncRNA antagonists.

### FIELD OF THE INVENTION

The present invention relates to functional analysis of microRNAs (miRNAs) or other short noncoding RNAs (ncRNAs). One method for analysing the function of miRNAs is to introduce miRNA sequestrating/antagonising agents, typically miRNA complementary oligonucleotides, and subsequently observe the resulting phenotype when the miRNA of interest is antagonised. The present invention involves the use of two or more sequence distinct miRNA antagonising oligomeric compounds, which enable the reagent redundancy experiments to reduce the risk of reporting false positive effects of miRNA/ncRNA antagonists.

### BACKGROUND OF THE INVENTION

### MicroRNAs

The expanding inventory of international sequence databases and the concomitant sequencing of nearly 200 genomes representing all three domains of life—bacteria, archea, and eukaryota—have been the primary drivers in the process of deconstructing living organisms into comprehensive molecular catalogs of genes, transcripts, and proteins. The importance of the genetic variation within a single species has become apparent, extending beyond the completion of genetic blueprints of several important genomes, culminating in the publication of the working draft of the human genome sequence in 2001 (Lander, Linton, Birren et al., 2001 Nature 409: 860-921; Venter, Adams, Myers et al., 2001 Science 291: 1304-1351; Sachidanandam, Weissman, Schmidt at al., 2001 Nature 409: 928-933). On the other hand, the increasing number of detailed, large-scale molecular analyses of transcription originating from the human and mouse genomes along with the recent identification of several types of non-protein-coding RNAs, such as small nucleolar RNAs, siRNAs, miRNAs and antisense RNAs, indicate that the transcriptomes of higher eukaryotes are much more complex than originally anticipated (Wong et al. 2001, Genome Research 11: 1975-1977; Kampa et al. 2004, Genome Research 14: 331-342).

As a result of the Central Dogma: ‘DNA makes RNA, and RNA makes protein’, RNAs have been considered as simple molecules that just translate the genetic information into protein. Recently, it has been estimated that although most of the genome is transcribed, almost 97% of the genome does not encode proteins in higher eukaryotes, but putative, non-coding RNAs (Wong et al. 2001, Genome Research 11: 1975-1977). The non-coding RNAs appear to be particularly well suited for regulatory roles that require highly specific nucleic acid recognition. Therefore, the view of RNA is rapidly changing from the merely informational molecule to comprise a wide variety of structural, informational and catalytic molecules in the cell.

Recently, a large number of small non-coding RNA genes have been identified and designated as miRNAs (for review, see Ke et al. 2003, Curr. Opin. Chem. Biol. 7:516-523). The first miRNAs to be discovered were the lin-4 and let-7 that are heterochronic switching genes essential for the normal temporal control of diverse developmental events (Lee et al. 1993, Cell 75:843-854; Reinhart et al. 2000, Nature 403: 901-906) in the roundworm C. elegans. miRNAs have been evolutionarily conserved over a wide range of species and exhibit diversity in expression profiles, suggesting that they occupy a wide variety of regulatory functions and exert significant effects on cell growth and development (Ke et al. 2003, Curr. Opin. Chem. Biol. 7:516-523). Recent work has shown that miRNAs can regulate gene expression at many levels, representing a novel gene regulatory mechanism and supporting the idea that RNA is capable of performing similar regulatory roles as proteins. Understanding this RNA-based regulation will help us to understand the complexity of the genome in higher eukaryotes as well as understand the complex gene regulatory networks.

miRNAs are 19-25 nucleotide (nt) RNAs that are processed from longer endogenous hairpin transcripts (Ambros et al. 2003, RNA 9: 277-279). To date more than 6000 miRNAs have been identified in humans, worms, fruit flies and plants according to the miRNA registry database release 11.0 in April 2008, hosted by Sanger Institute, UK, and many miRNAs that correspond to putative genes have also been identified. Some miRNAs have multiple loci in the genome (Reinhart et al. 2002, Genes Dev. 16: 1616-1626) and occasionally, several miRNA genes are arranged in tandem clusters (Lagos-Quintana et al. 2001, Science 294: 853-858).

The combined characteristics of miRNAs characterized to date (Ke et al. 2003, Curr. Opin. Chem. Biol. 7:516-523; Lee et al. 1993, Cell 75:843-854; Reinhart et al. 2000, Nature 403: 901-906) can be summarized as:
1. miRNAs are single-stranded RNAs of about 19-25 nt that regulate the expression, stability, and/or translation into protein of complementary messenger RNAs.
2. They are cleaved from a longer endogenous double-stranded hairpin precursor by the enzyme Dicer.
3. miRNAs match precisely the genomic regions that can potentially encode precursor RNAs in the form of double-stranded hairpins.
4. miRNAs and their predicted precursor secondary structures are phylogenetically conserved.

Several lines of evidence suggest that the enzymes Dicer and Argonaute are crucial participants in miRNA biosynthesis, maturation, and function (Grishok et al. 2001, Cell 106: 23-24). Mutations in genes required for miRNA biosynthesis lead to genetic developmental defects, which are, at least in part, derived from the role of generating miRNAs. The current view is that miRNAs are cleaved by Dicer from the hairpin precursor in the form of duplex, initially with 2 or 3 nt overhangs in the 3′ ends, and are termed pre-miRNAs. Cofactors join the pre-miRNP and unwind the pre-miRNAs into single-stranded miRNAs, and pre-miRNP is then transformed to miRNP. miRNAs can recognize regulatory targets while part of the miRNP complex. There are several similarities between miRNP and the RNA-induced silencing complex, RISC, including similar sizes and both containing RNA helicase and the PPD proteins. It has therefore been proposed that miRNP and RISC are the same RNP with multiple functions (Ke et al. 2003, Curr. Opin. Chem. Biol. 7:516-523). Different effectors direct miRNAs into diverse pathways. The structure of pre-miRNAs is consistent with the observation that 22 nt RNA duplexes with 2 or 3 nt overhangs at the 3′ ends are beneficial for reconstitution of the protein complex and might be required for high affinity binding of the short RNA duplex to the protein components (for review, see Ke et al. 2003, Curr. Opin. Chem. Biol. 7:516-523).

Growing evidence suggests that miRNAs play crucial roles in eukaryotic gene regulation. The first miRNA genes to be discovered, lin-4 and let-7, base-pair incompletely to repeated elements in the 3′ untranslated regions (UTRs) of other heterochronic genes, and regulate the translation directly and negatively by antisense RNA-RNA interaction (Lee et al. 1993, Cell 75:843-854; Reinhart et al. 2000, Nature 403: 901-906). Other miRNAs are thought to interact with target mRNAs by limited complementary and suppressed translation as well (Lagos-Quintana et al. 2001, Science 294: 853-858; Lee and Ambros 2001, Science 294: 858-862). Many studies have shown, however, that given a perfect complementarity between miRNAs and their target RNA, could lead to target RNA degradation rather than inhibit translation (Hutvagner and Zamore 2002, Science 297: 2056-2060), suggesting that the degree of complementarity determines their functions. By identifying sequences with near complementarity, several targets have been predicted, most of which appear to be potential transcriptional factors that are crucial in cell growth and development. The high percentage of predicted miRNA targets acting as developmental regulators and the conservation of target sites suggest that miRNAs are involved in a wide range of organism development and behaviour and cell fate decisions (for review, see Ke et al. 2003, Curr. Opin. Chem. Biol. 7:516-523). For example, John et al. 2004 (PLoS Biology 2: e363) used known mammalian miRNAs to scan the 3′ untranslated regions (UTRs) from human, mouse and rat genomes for potential miRNA target sites using a scanning algorithm based on sequence complementarity between the mature miRNA and the target site, binding energy of the miRNA:mRNA duplex and evolutionary conservation. They identified a total of 2307 target mRNAs conserved across the mammals with more than one target site at 90% conservation of target site sequence and 660 target genes at 100% conservation level. Scanning of the two fish genomes; Danio rerio (zebrafish) and Fugu rubripes (Fugu) identified 1000 target genes with two or more conserved miRNA sites between the two fish species (John et al. 2004 PLoS Biology 2: e363). Among the predicted targets, particularly interesting groups included mRNA encoding transcription factors, components of the miRNA machinery, other proteins involved in the translational regulation as well as components of the ubiquitin machinery. Wang et al. 2004 (Genome Biology 5:R65) have developed and applied a computational algorithm to predict 95 Arabidopsis thaliana miRNAs, which included 12 known ones and 83 new miRNAs. The 83 new miRNAs were found to be conserved with more than 90% sequence identity between the Arabidopsis and rice genomes. Using the Smith-Waterman nucleotide-alignment algorithm to predict mRNA targets for the 83 new miRNAs and by focusing on target sites that were conserved in both Arabidopsis and rice, Wang et al. 2004 (Genome Biology 5:R65) predicted 371 mRNA targets with an average of 4.8 targets per miRNA. A large proportion of these mRNA targets encoded proteins with transcription regulatory activity.

MiRNAs are found in all multicellular organisms and have crucial function in tuning the cellular RNA and protein expression. One method for analysing function of miRNAs is to introduce miRNA sequestrating/antagonising agents, typically miRNA complementary oligonucleotides, and subsequently observe the resulting phenotype when the miRNA of interest is antagonised.

### Off-Targeting

An inherent problem of introducing oligonucleotides into biological systems is that such agents cross react with other complementary oligomeric compounds such as mRNAs. This secondary effect may cause phenotypic reactions, also called false positives, which can be misinterpreted as a specific miRNA antagonising effect, although caused by cross reaction with other RNAs or DNAs.

In studies of gene function cellular introduction of siRNAs that specifically targets and triggers degradation of a complementary mRNA target is a very powerful technique.

However, recent experiments have shown that siRNAs are not entirely specific and also affects expression of a larger number of other genes that it was not designed to target. Such mistargeting is also referred to as off-target effects. Off-target effects can be a result of both sequence independent and sequence specific effects of the introduced siRNA. Sequence independent off-target effects can often be addressed by the use of several negative control oligos of random sequence. To rule out that phenotypic effects are due to sequence dependent off-targeting, several sequence distinct siRNAs targeting the same mRNA of interest are employed—this technique is referred to as reagent redundancy experiments, as described in Echeverri, C. J. et al, Nature Methods, vol. 3, No. 10, 777-779 (2006). To trust a phenotypic result of targeting a given mRNA, the phenotype should appear with all of these siRNAs independently of primary sequence.

As an alternative to the use of siRNAs, functional analysis of mRNAs can also be carried out using mRNA targeting oligonucleotides e.g. gapmers complementary to the mRNA. Such oligonucleotides may also result in some degree of sequence specific off-targeting effect, though this has been less investigated than with siRNAs.

Knockdown of miRNA

In functional studies of miRNAs antagonising oligonucleotides have been successful in down regulating the complementary miRNA. To generate an efficient antisense effect of the antagonising oligo the duplex between the miRNA and the antisense oligonucleotide are required to form a relatively stable duplex. To achieve sufficient stability in the miRNA-oligonucleotide antagonised duplex, oligonucleotides complementary to the entire miRNA sequence have been used. Such oligonucleotides can be of various chemical composition; e.g. 2′OMe and LNA-DNA mixmers/chimeras, as described in Kurreck, Jens, Eur. J. Biochem. 270, 1628-1644 (2003). Both 2′OMe and LNA-DNA mixmers/chimeras have shown to be effective. However, using oligonucleotides complementary to the full-length of the miRNA does not leave room for reagent redundancy experiments, as it is not possible to find a sequence distinct miRNA antagonising oligonucleotide targeting the same miRNA.

In the present invention we demonstrate that LNA containing miRNA antagonising oligonucleotides can be designed as short as 9meric compounds. The use of short oligonucleotides allows design of two or more sequence specific oligonucleotides targeting the same 19-24mer miRNA (or other short RNA), thereby allowing reagent redundancy experiments not previously achievable with prior techniques. As an alternative to 9mer compounds it is possible to use 9-15meric compounds which may be largely sequence specific, only sharing a short sequence stretch complementary to the central part of the miRNA. Alternatively to targeting the mature miRNA reagent redundancy experiments can also be carried out by targeting the precursors of the mature miRNA e.g. PremiR and PrimiR. Due to their transient nature targeting of the precursor molecules is likely to be less efficient than targeting of the mature forms.

The invention features methods of determining an off-target effect and whether binding of an antagonising oligonucleotide to a non-coding RNA, such as a miRNA, such as a mature miRNA, is associated with any unintended effects or false positives, such as off-target effects, immune response activation and/or non-specific gene silencing.

In one embodiment a method of determining an off-target effect induced by an antagonising oligonucleotide on an eukaryotic cell expressing a target non-coding RNA, such as a miRNA, such as a mature miRNA, comprises determining a phenotype of the eukaryotic cell after subjecting the cell to the antagonising oligonucleotide and one or more additional antagonising oligonucleotides of the invention, binding to a region within the target non-coding RNA, such as a miRNA, such as a mature miRNA.

Thus, in one embodiment the present invention provides a method of determining whether a phenotype induced by an antagonising oligonucleotide for a target non-coding RNA, such as a miRNA, such as a mature miRNA, is a false positive, said method comprising:

(a) introducing a first antagonising oligonucleotide into a first target cell, wherein said first antagonising oligonucleotide comprises a recognition sequence hybridizing to a first region of said target RNA;
(b) measuring a phenotype in said first target cell after (a);
(c) introducing a second antagonising oligonucleotide into a second target cell, wherein said second antagonising oligonucleotide comprises a recognition sequence hybridizing to a second region of said target RNA;
(d) measuring a phenotype in said second target cell after (c); and
(e) comparing the phenotype in said first target cell with the phenotype in said second target cell,
wherein, if the phenotype in said first target cell is similar to the phenotype in said second target cell, the phenotype observed in said first target cell is a false positive. In preferred embodiments the antagonising oligonucleotides are no longer than 15 nucleotides in length, such as 9-15 nucleotides in length, or such as 9-10 nucleotides in length. In another preferred embodiment the first region of the target RNA comprises the 3′ end and the second region of the target RNA comprises the 5′ end.

In a preferred embodiment, the determining the phenotype comprises determining the expression levels by array analysis as described herein of a plurality of different genes, such as at least 5 different genes, such as at least 10 different genes, such as at least 100 different genes, such as at least 1000 different genes, such as at least 10,000 different genes, or such as at least 25,000 different genes, and/or their translation products.

As off-targeting can induce measurable phenotypes, including potential toxicity, and problems in data interpretation, it represents a large impediment for therapeutic and phenotypic screening applications.

The ability to use several sequence specific oligonucleotide compounds for targeting of short RNAs can be used in optimization of oligonucleotide therapeutics. E.g. testing of various therapeutic compounds (short oligonucleotides) targeting the same short RNA, in order to obtain the short oligonucleotide compound with the least serious side effects.

In conclusion, a challenge in functional analysis of non-coding RNAs, such as miRNAs, such as mature miRNAs, and the exploitation of antagonising oligonucleotides as a gene knockdown tool for research and in therapy is the ability of antagonising oligonucleotides to target multiple target nucleotides in an undesired manner. The present invention provides the development of novel antagonising oligonucleotide compositions, providing an accurate and highly sensitive solution to specifically block a particular target non-coding RNA, such as a miRNA, such as a mature miRNA, useful for determining the level of off-targeting.

### FIGURES

FIG. 1. HeLa cells co-transfected with pMIR-21 and the indicated oligonucleotides. The diagram shows fold up regulation; FL (Fluorescence); RLU (Rleative Light Unit); RL (Renilla Luciferase). The “no target” column is the expression level of the parental reporter vector with no miR-21 target site.

FIG. 2. HeLa cells co-transfected with pMIR-24 and the indicated oligonucleotides. The diagram shows fold up regulation; FL (Fluorescence); RLU (Rleative Light Unit); RL (Renilla Luciferase). The “no target” column is the expression level of the parental reporter vector with no miR-24 target site.

### SUMMARY OF THE INVENTION

In one aspect, the invention provides antagonising oligonucleotides including a high affinity nucleic acid analog, e.g. a Locked Nucleic Acid (LNA), and binding to a region of a target non-coding RNA, such as a miRNA, such as a mature miRNA. The antagonising oligonucleotide binds, for example, to the 3′ end or the 5′ end of the target non-coding RNA, such as a miRNA, such as a mature miRNA. The antagonising oligonucleotide is, for example, from 5 to 15 nucleotides, e.g. from 8 to 13 nucleotides.

In certain preferred embodiments, the antagonising oligonucleotide includes a plurality of high affinity nucleotide analogs of the same or different types. For example, the antagonising oligonucleotide may include up to 80%, e.g., up to 75, 70, 65, 60, 55, 50, 45, 40, 35, 30, 25, or 20%, of the high affinity nucleic acid analog or the high affinity nucleic acid analog, e.g., LNA, optionally in combination with one or more additional analogs, e.g., 2′ OMe. The plurality of analogs may be disposed so that no more than four naturally occurring nucleotides occur in linear sequence.

A high affinity nucleic acid analog may or may not be disposed at the 3′ or 5′ end of the nucleic acid. In a preferred embodiment, the antagonising oligonucleotide is RNase resistant. In other embodiments, the high affinity nucleic acid analog(s) are not disposed in regions capable of forming auto-dimers or intramolecular complexes.

In preferred embodiments wherein the target non-coding RNA is a mature miRNA, the nucleic acid does not prevent production of the mature miRNA from its corresponding pri- or pre-miRNA.

The binding of the nucleic acid to the region desirably reduces the binding of the mature miRNA to its target site, by at least 50%, e.g. by at least 55%, 60%, 65%, 70%, 75%, 80%, 85%, or 90%. Alternatively, the nucleic acid binds to the region with a lower Kd than the miRNA in vivo. The nucleic acid may also have an increase in binding affinity to the region as determined by an increase in Tm of at least 2° C., compared to the naturally occurring RNA complement of the region.

The invention also features a method of treating a disease caused by binding of an miRNA to a target site by contacting a subject with two or more antagonising oligonucleotides of the invention in an amount sufficient to reduce binding of the miRNA at the target site, by at least 50%, e.g. by at least 55%, 60%, 65%, 70%, 75%, 80%, 85%, 90%, or 95%.

The use of two distinct oligonucleotides for antisense inhibition of a target oligonucleotide as illustrated in Scheme 1 with the targeting of hsa-miR-21 as an example. This technique can be used to carry out reagent redundancy experiments to reduce the risk of reporting false positive phenotypes due to off-target effect.

### Example of Two Oligo Knockdown:

SCHEME 1
Illustration of how two distinct oligo can target
the same RNA target. In this example illustrated
with the targeting of hsa-miR-21 with two short
LNA oligonucleotides.

| | | |
|---|---|---|
| Hsa-mir-21: | UAGCUUAUCAGACUGAUGUUGA | (SEQ ID NO.: 1) |
| Anti mir- | ATCGAATAGTCTGACTACAACT | (SEQ ID NO.: 2) |
| 21: | | |
| 5′ Knock- | ATCGAATAGT | (SEQ ID NO.: 3) |
| down: | | |
| 3′ Knock- | TGACTACAA | (SEQ ID NO.: 4) |
| down: | | |

In a particular embodiment of the invention oligonucleotides are referred to as “oligonucleotide compositions”.

“Oligonucleotide compositions” are oligonucleotides wherein at least one monomer is a non-natural nucleotide also designated a “modified monomer unit”, which preferably is a LNA monomer as defined below and the remaining monomers are natural nucleotides. Preferred LNA monomers are oxy-LNA, alpha-LNA and amino-LNA as defined below.

An example of an oligonucleotide composition of the invention and the corresponding reference oligonucleotide composition are shown in Table 1.

TABLE 1

| | | |
|---|---|---|
| Target sequence | 5′-uagcuuaucagacugauguga-3′ | (SEQ ID NO.: 1) |
| (miRNA | | |
| hsa-miR-21) | | |
| miR-21 3′ | 5′-AAmCATmCAGT-3′ | (SEQ ID NO.: 5) |
| miR-21-5′ | 5′-TGATAAGmCTA-3′ | (SEQ ID NO.: 6) |

### Abbreviations:

Capital G, A, T, or mC letters indicates oxy-LNA
Lowercase indicates natural DNA/RNA
mC indicates 5-methylcytosine

Oligonucleotide compositions shown in Table 1 is based on the reverse complementary sequence of the miRNA hsa-miR-21 (miRBase, Version 10.1, accession number MIMAT0000076).

The present invention also provides a kit for sequence specific inactivation of intracellular nucleic acids. The present invention also provides a kit for introduction of nucleic acids to a cell.

The present invention also allows conjugation of the oligonucleotide compounds to functional groups that improve the properties of the oligonucleotide. Such compounds could enhance cellular uptake, bio-distribution, -availability and -stability, pharmacokinetics etc. Furthermore, attachment of various labels (e.g. fluorophores) to the oligonucleotide allows monitoring delivery of the oligonucleotides to cells and animals.

For the kits according to the invention, the reaction body is preferably a solid support material, e.g. selected from borosilicate glass, soda-lime glass, polystyrene, polycarbonate, polypropylene, polyethylene, polyethyleneglycol terephthalate, polyvinylacetate, polyvinylpyrrolidinone, polymethylmethacrylate and polyvinylchloride, preferably polystyrene and polycarbonate. The reaction body may be in the form of a specimen tube, a vial, a slide, a sheet, a film, a bead, a pellet, a disc, a plate, a ring, a rod, a net, a filter, a tray, a microtitre plate, a stick, or a multi-bladed stick.

A written instruction sheet stating the optimal conditions for use of the kit typically accompanies the kits.

LNA substituted oligomers are preferably chemically synthesized using commercially available methods and equipment as described in the art (Tetrahedron 54: 3607-30, 1998). For example, the solid phase phosphoramidite method can be used to produce short LNA probes (Caruthers, et al., Cold Spring Harbor Symp. Quant. Biol. 47:411-418, 1982, Adams, et al., J. Am. Chem. Soc. 105: 661 (1983).

Suitable samples of target nucleic acid molecules may comprise a wide range of eukaryotic cells. The methods are thus applicable systemic delivery in metazoans. To tissue culture of animal cells (e.g., fibroblasts, lymphocytes, embryonic stem cells, osteoblasts, neurons, oocytes) any type of tissue biopsy/explant (e.g. a muscle biopsy, a liver biopsy, a kidney biopsy, a bladder biopsy, a bone biopsy, a cartilage biopsy, a skin biopsy, a pancreas biopsy, a biopsy of the intestinal tract, a thymus biopsy, a mammae biopsy, a uterus biopsy, a testicular biopsy, an eye biopsy or a brain biopsy), plant cells and intact plants and algae.

### DETAILED DESCRIPTION OF THE INVENTION

A probe according to the present invention preferably comprises from about 5 to about 15 monomer subunits. It is more preferred that such probes comprise from about 8 to about 13 monomer subunits.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure. However, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

A further preferred modification includes Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage is preferably a methylene (—CH2-)ₙ group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards T-exonucleolytic degradation and good solubility properties.

Novel types of LNA-modified oligonucleotides, as well as the LNAs, are useful in a wide range of diagnostic and therapeutic applications. Among these are antisense applications, PCR applications, strand-displacement oligomers, substrates for nucleic acid polymerases and generally as nucleotide based drugs.

Potent and nontoxic antisense oligonucleotides containing LNAs have been described (Wahlestedt et al., Proc. Natl. Acad. Sci. U.S.A., 2000, 97, 5633-5638.) The authors have demonstrated that LNAs confer several desired properties to antisense agents. LNA/DNA copolymers were not degraded readily in blood serum and cell extracts. LNA/DNA copolymers exhibited potent antisense activity in assay systems as disparate as G-protein-coupled receptor signalling in living rat brain and detection of reporter genes in Escherichia coli.

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

In various embodiments, a nucleic acid of the invention specifically includes one or more of 2′-O-methyl-modified nucleic acids (2′-OMe), 2′-O-(2-methoxyethyl)-modified nucleic acids (2′-MOE), 2′-Deoxy-2′-fluoro-13-D-arabinoic acid (FANA), Cyclohexene nucleic acids (CeNA), Hexitol nucleic acids (HNA) and analogs thereof, Intercalating Nucleic Acids (INA), 2′-O,4′-C-Ethylene-bridged-Nucleic Acids (ENA), and peptide nucleic acid (PNA). In other embodiments, a nucleic acid of the invention does not include 2′-O-methyl-modified nucleic acids (2′-OMe); a nucleic acid of the invention does not include 2′-O-(2-methoxyethyl)-modified nucleic acids (2′-MOE); a nucleic acid of the invention does not include 2′-Deoxy-2′-fluoro-β-D-arabinoic acid (FANA); a nucleic acid of the invention does not include Cyclohexene nucleic acids (CeNA); a nucleic acid of the invention does not include Hexitol nucleic acids (HNA) or analogs thereof; a nucleic acid of the invention does not include Intercalating Nucleic Acids (INA); a nucleic acid of the invention does not include 2′-O,4′-C-Ethylene-bridged-Nucleic Acids (ENA); and/or a nucleic acid of the invention does not include peptide nucleic acids (PNA).

The invention also features a pharmaceutical composition including two or more antagonising oligonucleotides of the invention and a pharmaceutically acceptable excipient. Pharmaceutical compositions may be used in treatment of diseases associated with miRNA, as described herein. The invention also includes a diagnostic kit including two or more antagonising oligonucleotides of the invention. The diagnostic kits may be employed to diagnose a disease associated with an miRNA, to prognose a subject having a disease associated with an miRNA, to determine the risk of a subject to develop a disease associated with an miRNA, or to determine the efficacy of a particular treatment for a disease associated with an miRNA. The antagonising oligonucleotides of the invention may further be used as research tools and in drug screening, as described herein.

Some preferred miRNAs are those associated with cancer, heart disease, cardiovascular disease, neurological diseases such as Parkinson's disease, Alzheimer's, spinal muscular atrophy and X mental retardation, atherosclerosis, postangioplasty restenosis, transplantation arteriopathy, stroke, viral infection, psoriasis, metabolic disease, diabetes mellitus, and diabetic nephropathy.

The invention further features a method of inhibiting the binding of an miRNA to a target site by contacting two or more nucleic acids of the invention with a cell expressing the target site. The contacting may occur in vitro, e.g., in drug screening, or in vivo, e.g., in therapy.

Such methods may be employed diagnostically, as described.

The probes of the invention are useful for research, diagnostics as well as therapy, because these compounds can be prepared to hybridize to nucleic acids encoding miRNA/piRNA/shRNA and aRNA (and other small RNAs) regulating the encoding nucleic acids.

### Methods for Determining Biological State and Biological Response.

This invention provides methods comprising determining response profiles, such as changes of phenotypes, of specific blocking of siRNA perturbation. The measured responses can be measurements of cellular constituents in a cell or organism or responses of a cell or organism to a specific blocking of siRNA perturbation. The cell sample can be of any organism in which RNA interference can occur, e.g., eukaryote, mammal, primate, human, non-human animal such as a dog, cat, horse, cow, mouse, rat, Drosophila, C. elegans, etc., plant such as rice, wheat, bean, tobacco, etc., and fungi. The cell sample can be from a diseased or healthy organism, or an organism predisposed to disease. The cell sample can be of a particular tissue type or development stage and subjected to a particular siRNA perturbation. One of skill in the art would appreciate that this invention is not limited to the following specific methods for measuring the phenotypes, such as expression profiles and responses, of a biological system.

### Transcript Assays Using Microarrays.

One aspect of the invention provides polynucleotide probe arrays for simultaneous determination of the expression levels of a plurality of genes and methods for designing and making such polynucleotide probe arrays. The expression level of a nucleotide sequence in a gene can be measured by any high throughput techniques. However measured, the result is either the absolute or relative amounts of transcripts or response data, including but not limited to values representing abundance ratios. Preferably, measurement of the expression profile is made by hybridization to transcript arrays, such as described in PCT patent application no. WO 2005/18534.

The relative abundance of an mRNA and/or an exon expressed in an mRNA in two cells or cell lines is scored as different (i.e., the abundance is different in the two sources of mRNA tested) or as identical (i.e., the relative abundance is the same). As used herein, a difference between the two sources of RNA of at least a factor of about 25% (i.e., RNA is 25% more abundant in one source than in the other source), more usually about 50%, even more often by a factor of about 2 (i.e., twice as abundant), 3 (three times as abundant), or 5 (five times as abundant) is scored as different. Present detection methods allow reliable detection of difference of an order of about 3-fold to about 5-fold, but more sensitive methods are expected to be developed.

### Other Methods of Transcriptional State Measurement.

The transcriptional state of a cell may be measured by other gene expression technologies known in the art. Several such technologies produce pools of restriction fragments of limited complexity for electrophoretic analysis, such as methods combining double restriction enzyme digestion with phasing primers (see, e.g., European Patent O 534858 A1, filed Sep. 24, 1992, by Zabeau et al.), or methods selecting restriction fragments with sites closest to a defined mRNA end (see, e.g., Prashar et al., 1996, Proc. Natl. Acad. Sci. USA 93:659-663). Other methods statistically sample cDNA pools, such as by sequencing sufficient bases (e.g., 20-50 bases) in each of multiple cDNAs to identify each cDNA, or by sequencing short tags (e.g., 9-10 bases) that are generated at known positions relative to a defined mRNA end (see, e.g., Velculescu, 1995, Science 270:484-487).

### Measurement of Other Aspects of the Biological State.

In various embodiments of the present invention, aspects of the biological state other than the transcriptional state, such as the translational state, the activity state, or mixed aspects can be measured to produce the measured signals to be analyzed according to the invention. Thus, in such embodiments, gene expression data may include translational state measurements or even protein expression measurements. In fact, in some embodiments, rather than using gene expression interaction maps based on gene expression, protein expression interaction maps based on protein expression maps are used.

### Embodiments Based on Translational State Measurements.

Measurement of the translational state may be performed according to several methods. For example, whole genome monitoring of protein (i.e., the “proteome,” Goffeau et al., 1996, Science 274:546-567; Aebersold et al., 1999, Nature Biotechnology 10:994-999) can be carried out by constructing a microarray in which binding sites comprise immobilized, preferably monoclonal, antibodies specific to a plurality of protein species encoded by the cell genome (see, e.g., Zhu et al., 2001, Science 293:2101-2105; MacBeath et al., 2000, Science 289:1760-63; de Wildt et al., 2000, Nature Biotechnology 18:989-994). Preferably, antibodies are present for a substantial fraction of the encoded proteins, or at least for those proteins relevant to the action of an siRNA of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In a preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated and measured by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of proteins: A Practical Approach, IRL Press, New York; Shevchenko et al., 1996, Proc. Natl. Acad. Sci. USA 93:1440-1445; Sagliocco et al., 1996, Yeast 12:1519-1533; Lander, 1996, Science 274:536-539; and Beaumont et al., Life Science News 7, 2001, Amersham Pharmacia Biotech. The resulting electropherograms can be analyzed by numerous techniques, including mass spectrometric techniques, Western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to an siRNA and/or a blocking oligo of the invention, or in cells modified by, e.g., deletion or over-expression of a specific gene.

### Embodiments Based on Other Aspects of the Biological State.

The methods of the invention are applicable to any cellular constituent that can be monitored. In particular, where activities of proteins can be measured, embodiments of this invention can use such measurements. Activity measurements can be performed by any functional, biochemical, or physical means appropriate to the particular activity being characterized. Where the activity involves a chemical transformation, the cellular protein can be contacted with the natural substrate(s), and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In alternative and non-limiting embodiments, phenotype data may be formed of mixed aspects of the biological state of a cell. Phenotype data can be constructed from, e.g., changes in certain mRNA abundances, changes in certain protein abundances, and changes in certain protein activities.

### DEFINITIONS

In the context of this invention, the terms “probe”, “oligonucleotide probe” or “oligomeric compound” refer to polymeric structures capable of hybridizing to a region of a nucleic acid molecule. These terms include oligonucleotides, oligonucleosides, oligonucleotide analogs, modified oligonucleotides and oligonucleotide mimetics. The compounds can be prepared to be linear or circular and may include branching. They can be prepared single stranded or double stranded and may include overhangs. In general an oligomeric compound comprises a backbone of linked monomeric subunits where each linked monomeric subunit is directly or indirectly attached to a heterocyclic base moiety. The linkages joining the monomeric subunits, the monomeric subunits and the heterocyclic base moieties can be variable in structure giving rise to a plurality of motifs for the resulting oligomeric compounds including hemimers, gapmers and chimeras.

In the context of this invention, the term “oligonucleotide” refers to an oligomer or polymer of ribonucleic acid (RNA) or deoxyribonucleic acid (DNA). This term includes oligonucleotides composed of naturally-occurring nucleobases, sugars and covalent internucleoside linkages. The terms “oligonucleotide analog” and “modified oligonucleotide” refers to oligonucleotides that have one or more non-naturally occurring portions which function in a similar manner to oligonucleotides. Such modified or substituted oligonucleotides are often preferred over native forms because of desirable properties such as, for example, enhanced cellular uptake, enhanced affinity for nucleic acid target, increased or decreased specificity of duplex formation and increased stability in the presence of nucleases.

The term “Recognition sequence” refers to a nucleotide sequence that is complementary to a region within the target nucleotide sequence essential for sequence-specific hybridization between the target nucleotide sequence and the recognition sequence.

The term “label” as used herein refers to any atom or molecule which can be used to provide a detectable (preferably quantifiable) signal, either directly or indirectly and which can be attached to a nucleic acid or protein or to any atom or molecule.

Labels may provide signals detectable by fluorescence, radioactivity, colorimetric, X-ray diffraction or absorption, magnetism, enzymatic activity, and the like or may provide recognition sites for labelling reagents such as antibodies or nucleic acids having detectable labels (“indirect labelling”; “indirect detection”).

Labels may also comprise ligands. In the present context “ligand” means something, which binds. Ligands comprise biotin and functional groups such as: aromatic groups (such as benzene, pyridine, naphtalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C₁-C₂₀ alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-13-alanine, polyglycine, polylysine, peptides, oligo/polysaccharides, oligo/polyphosphates, toxins, antibiotics, cell poisons, and steroids, and also “affinity ligands”, i.e. functional groups or biomolecules that have a specific affinity for sites on particular proteins, antibodies, poly- and oligosaccharides, and other biomolecules.

Further examples of functional parts of labels are biotin, digoxigenin, fluorescent groups (groups which are able to absorb electromagnetic radiation, e.g. light or X-rays, of a certain wavelength, and which subsequently reemits the energy absorbed as radiation of longer wavelength; illustrative examples are DANSYL (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO(N-oxyl-2,2,6,6-tetramethylpiperidine), dinitrophenyl, acridines, coumarins, Cy3 and Cy5 (trademarks for Biological Detection Systems, Inc.), erythrosine, coumaric acid, umbelliferone, Texas red, rhodamine, tetramethyl rhodamine, Rox, 7-nitrobenzo-2-oxa-1-diazole (NBD), pyrene, fluorescein, Europium, Ruthenium, Samarium, and other rare earth metals), radio isotopic labels, chemiluminescence labels (labels that are detectable via the emission of light during a chemical reaction), spin labels (a free radical (e.g. substituted organic nitroxides) or other paramagnetic probes (e.g. Cu²⁺, Mg²⁺) bound to a biological molecule being detectable by the use of electron spin resonance spectroscopy). Especially interesting examples are biotin, fluorescein, Texas Red, rhodamine, dinitrophenyl, digoxigenin, Ruthenium, Europium, Cy5, Cy5.5, Cy3, etc.

As used herein, the terms “nucleic acid”, “polynucleotide” and “oligonucleotide” refer to primers, probes, oligomer fragments to be detected, oligomer controls and unlabelled blocking oligomers and shall be generic to polydeoxyribonucleotides (containing 2-deoxy-D-ribose), to polyribonucleotides (containing D-ribose), and to any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA. The oligonucleotide is comprised of a sequence of approximately at least 3 nucleotides, preferably at least about 6 nucleotides, and more preferably at least about 8-30 nucleotides corresponding to a region of the designated target nucleotide sequence. “Corresponding” means identical to or complementary to the designated sequence. The oligonucleotide is not necessarily physically derived from any existing or natural sequence but may be generated in any manner, including chemical synthesis, DNA replication, reverse transcription or a combination thereof.

The term “nucleic acid” intend a polynucleotide of genomic DNA or RNA, cDNA, semi synthetic, or synthetic origin which, by virtue of its origin or manipulation. Because mononucleotides are reacted to make oligonucleotides in a manner such that the 5′-OH of one mononucleotide pentose ring is attached to the 3′ oxygen of its neighbour in one direction via a phosphodiester linkage, an end of an oligonucleotide is referred to as the “5′ end” if its 5′-OH is not linked to the 3′ oxygen via a phosphodiester linkage of a mononucleotide pentose ring and as the “3′ end” if its 3′ oxygen is not linked to a 5′-OH of a subsequent mononucleotide pentose ring. As used herein, a nucleic acid sequence, even if internal to a larger oligonucleotide or having attached free phosphate groups, also may be said to have a 5′ and 3′ ends. When two different, non-overlapping oligonucleotides anneal to different regions of the same linear complementary nucleic acid sequence, the 3′ end of one oligonucleotide points toward the 5′ end of the other; the former may be called the “upstream” oligonucleotide and the latter the “downstream” oligonucleotide.

The complement of a nucleic acid sequence as used herein refers to an oligonucleotide which, when aligned with the nucleic acid sequence such that the 5′ end of one sequence is paired with the 3′ end of the other, is in “antiparallel association.” Bases not commonly found in natural nucleic acids may be included in the nucleic acids of the present invention include, for example, inosine and 7-deazaguanine. Complementarity may not be perfect; stable duplexes may contain mismatched base pairs or unmatched bases. Those skilled in the art of nucleic acid technology can estimate duplex stability empirically considering a number of variables including, for example, the length of the oligonucleotide, percent concentration of cytosine and guanine bases in the oligonucleotide, ionic strength, and incidence of mismatched base pairs.

Stability of a nucleic acid duplex is measured by the melting temperature, or “Tₘ”. The Tₘ of a particular nucleic acid duplex under specified conditions is the temperature at which half of the duplexes have disassociated.

The term “nucleobase” covers the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine (also termed “mC”), 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, Nucleic Acid Research, 25: 4429-4443, 1997. The term “nucleobase” thus includes not only the known purine and pyrimidine heterocycles, but also heterocyclic analogues. tautomers thereof. Further naturally and non naturally occurring nucleobases include those disclosed in U.S. Pat. No. 3,687,808; in chapter 15 by Sanghvi, in Antisense Research and Application, Ed. S. T. Crooke and B. Lebleu, CRC Press, 1993; in Englisch, et al., Angewandte Chemie, International Edition, 30: 613-722, 1991 (see, especially pages 622 and 623, and in the Concise Encyclopedia of Polymer Science and Engineering, J. I. Kroschwitz Ed., John Wiley & Sons, pages 858-859, 1990, Cook, Anti-Cancer DrugDesign 6: 585-607, 1991, each of which are hereby incorporated by reference in their entirety).

The term “nucleosidic base” or “nucleobase analogue” is further intended to include heterocyclic compounds that can serve as like nucleosidic bases including certain “universal bases” that are not nucleosidic bases in the most classical sense but serve as nucleosidic bases. Especially mentioned as a universal base is 3-nitropyrrole or a 5-nitroindole. Other preferred compounds include pyrene and pyridyloxazole derivatives, pyrenyl, pyrenylmethylglycerol derivatives and the like. Other preferred universal bases include, pyrrole, diazole or triazole derivatives, including those universal bases known in the art. Further exemplary modified bases are described in Guckian, et al., Journal of the American Chemical Society, 122: 2213-2222, 2000, EP 1 072 679 and WO 97/12896.

By “oligonucleotide,” “oligomer,” or “oligo” is meant a successive chain of monomers (e.g., glycosides of heterocyclic bases) connected via internucleoside linkages. The linkage between two successive monomers in the oligonucleotide consist of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O,O⁻)—, —P(O,OH)—, —PO(BH₃)—, —P(O,S⁻)—, —P(O,SH)—, —P(S,O⁻)—, —P(S,OH)—, P(S,S⁻) —, —P(S,SH)—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—NR^{H}—, —NR^{H}—CS—NR^{H}—, —NR^{H}—C(═NR^{H})—NR^{H}—, —NR^{H}—CO—CH₂—NR^{H}—, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N=(including R⁵ when used as a linkage to a succeeding monomer), —CH₂—O—NR^{H}—, —CO—NR^{H}—CH₂—, —CH₂—NR^{H}—CO—, —O—NR^{H}—CH₂—, —O—NR^{H}—, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—, —O—S(O)₂—CH₂—, —O—P(O,OH)—O—, —O—P(O,O⁻)—O—, —O—P(O,SH)—O—, —O—P(O,S⁻)—O—, O—P(S,OH)—O—, —O—P(S,O⁻)—O—, —O—P(S,SH)—O—, —O—P(S,S⁻)—O—, —S—P(O,OH)—O—, —S—P(O,O⁻)—O—, —S—P(O,SH)—O—, —S—P(O,S⁻)—O—, —S—P(S,OH)—O—, —S—P(S,O⁻)—O—, —S—P(S,S⁻)—O—, —S—P(S,SH)—O—, —O—P(O,O⁻)—S—, O—P(O,OH)—S—, —O—P(O,SH)—S—, —O—P(O,S⁻)—S—, —O—P(S,OH)—S—, —O—P(S,O⁻)—S—, —O—P(S,SH)—S—, —O—P(S,S⁻)—S—, —S—P(O,O⁻)—S—, —S—P(O,OH)—S—, —S—P(O,SH)—S—, —S—P(O,S⁻)—S—, S—P(S,OH)—S—, —S—P(S,O⁻)—S—, —S—P(S,SH)—S—, —S—P(S,S⁻)—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}—, —NR^{H}—P(O,OH)—O—, —O—P(O,NR^{H})—O—, —CH₂—P(O,OH)—O—, —O—P(O,OH)—CH₂—, and —O—Si(R″)₂—O—; among which —CH₂—CO—NR^{H}—, —CH₂—NR^{H}—O—, —S—CH₂—O—, —O—P(O,OH)—O—, —NR^{H}—P(O,OH)—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are especially desirable. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443. The left-hand side of the internucleoside linkage is bound to the 5-membered ring at the 3′-position, whereas the right-hand side is bound to the 5′-position of a preceding monomer.

By “LNA” or “LNA monomer” (e.g., an LNA nucleoside or LNA nucleotide) or an LNA oligomer (e.g., an oligonucleotide or nucleic acid) is meant a nucleoside or nucleotide analogue that includes at least one LNA monomer of formula (I), described infra, having the below described illustrative examples of modifications:

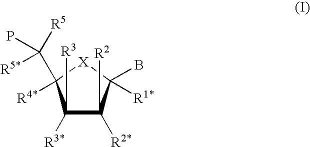

wherein X is selected from —O—, —S—, —N(R^{N})—, —C(R⁶R⁶*)—, —O—C(R⁷R⁷*)—, —C(R⁶R⁶*)—O—, —S—C(R⁷R⁷*)—, —C(R⁶R⁶*)—S—, —N(R^{N}*)—C(R⁷R⁷*)—, —C(R⁶R⁶*)—N(R^{N}*)—, and —C(R⁶R⁶*)—C(R⁷R⁷*).

B is selected from a modified base as discussed above e.g. an optionally substituted carbocyclic aryl such as optionally substituted pyrene or optionally substituted pyrenylmethylglycerol, or an optionally substituted heteroalicylic or optionally substituted heteroaromatic such as optionally substituted pyridyloxazole, optionally substituted pyrrole, optionally substituted diazole or optionally substituted triazole moieties; hydrogen, hydroxy, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands.

P designates the radical position for an internucleoside linkage to a succeeding monomer, or a 5′-terminal group, such internucleoside linkage or 5′-terminal group optionally including the substituent R⁵. One of the substituents R², R²*, R³, and R³* is a group P* which designates an internucleoside linkage to a preceding monomer, or a 2′/3′-terminal group. The substituents of R¹*, R⁴*, R⁵, R⁵*, R⁶, R⁶*, R⁷, R⁷*, R^{N}, and the ones of R², R²*, R³, and R³* not designating P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C=Q, wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R¹*, R², R²*, R³, R³*, R⁴*, R⁵, R⁵*, R⁶ and R⁶*, R⁷, and R⁷* which are present and not involved in P, P* or the biradical(s) together may form an associated biradical selected from biradicals of the same kind as defined before; the pair(s) of non-geminal substituents thereby forming a mono- or bicyclic entity together with (i) the atoms to which said non-geminal substituents are bound and (ii) any intervening atoms.

Each of the substituents R¹*, R², R²*, R³, R⁴*, R⁵, R⁵*, R⁶ and R⁶*, R⁷, and R⁷* which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di-(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR″)— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof.

LNA monomers as disclosed in PCT Publication WO 99/14226 are in general particularly desirable modified nucleic acids for incorporation into an oligonucleotide of the invention. Additionally, the nucleic acids may be modified at either the 3′ and/or 5′ end by any type of modification known in the art. For example, either or both ends may be capped with a protecting group, attached to a flexible linking group, attached to a reactive group to aid in attachment to the substrate surface, etc. Desirable LNA monomers and their method of synthesis also are disclosed in U.S. Pat. No. 6,043,060, U.S. Pat. No. 6,268,490, PCT Publications WO 01/07455, WO 01/00641, WO 98/39352, WO 00/56746, WO 00/56748 and WO 00/66604 as well as in the following papers: Morita et al., Bioorg. Med. Chem. Lett. 12(1):73-76, 2002; Hakansson et al., Bioorg. Med. Chem. Lett. 11(7):935-938, 2001; Koshkin et al., J. Org. Chem. 66(25):8504-8512, 2001; Kvaerno et al., J. Org. Chem. 66(16):5498-5503, 2001; Hakansson et al., J. Org. Chem. 65(17):5161-5166, 2000; Kvaerno et al., J. Org. Chem. 65(17):5167-5176, 2000; Pfundheller et al., Nucleosides Nucleotides 18(9):2017-2030, 1999; and Kumar et al., Bioorg. Med. Chem. Lett. 8(16):2219-2222, 1998.

When at least two LNA nucleotides are included in the oligonucleotide composition, these may be consecutive or separated by one or more non-LNA nucleotides. In one aspect, LNA nucleotides are alpha-L-LNA and/or xylo LNA nucleotides as disclosed in U.S. Pat. Nos. 7,053,207 and 7,084,125, resp.

Preferred LNA monomers, also referred to as “oxy-LNA” are LNA monomers which include bicyclic compounds as disclosed in PCT Publication WO 03/020739 wherein the bridge between R^{4′} and R^{2′} together designate —CH₂—O— or —CH₂—CH₂—O—.

Preferred LNA monomers, also referred to as “amino-LNA” are LNA monomers which include bicyclic compounds as claimed in U.S. Pat. No. 6,794,499 or U.S. Pat. No. 6,670,461 wherein the bridge between R^{4′} and R^{2′} together designate —CH₂—NH—.

Exemplary 5′, 3′, and/or 2′ terminal groups include —H, —OH, halo (e.g., chloro, fluoro, iodo, or bromo), optionally substituted aryl, (e.g., phenyl or benzyl), alkyl (e.g., methyl or ethyl), alkoxy (e.g., methoxy), acyl (e.g. acetyl or benzoyl), aroyl, aralkyl, hydroxy, hydroxyalkyl, alkoxy, aryloxy, aralkoxy, nitro, cyano, carboxy, alkoxycarbonyl, aryloxycarbonyl, aralkoxycarbonyl, acylamino, aroylamino, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, amidino, amino, carbamoyl, sulfamoyl, alkene, alkyne, protecting groups (e.g., silyl, 4,4′-dimethoxytrityl, monomethoxytrityl, or trityl(triphenylmethyl)), linkers (e.g., a linker containing an amine, ethylene glycol, quinone such as anthraquinone), detectable labels (e.g., radiolabels or fluorescent labels), and biotin.

It is understood that references herein to a nucleic acid unit, nucleic acid residue, LNA monomer, or similar term are inclusive of both individual nucleoside units and nucleotide units and nucleoside units and nucleotide units within an oligonucleotide.

The term “chemical moiety” refers to a part of a molecule. “Modified by a chemical moiety” thus refer to a modification of the standard molecular structure by inclusion of an unusual chemical structure. The attachment of said structure can be covalent or non-covalent.

The term “inclusion of a chemical moiety” in an oligonucleotide probe thus refers to attachment of a molecular structure. Such as chemical moiety include but are not limited to covalently and/or non-covalently bound minor groove binders (MGB) and/or intercalating nucleic acids (INA) selected from a group consisting of asymmetric cyanine dyes, DAPI, SYBR Green I, SYBR Green II, SYBR Gold, PicoGreen, thiazole orange, Hoechst 33342, Ethidium Bromide, 1-O-(1-pyrenylmethyl)glycerol and Hoechst 33258. Other chemical moieties include the modified nucleobases, nucleosidic bases or LNA modified oligonucleotides.

“High affinity nucleotide analogue” or “affinity-enhancing nucleotide analogue” refers to a non-naturally occurring nucleotide analogue that increases the “binding affinity” of an oligonucleotide probe to its complementary recognition sequence when substituted with at least one such high-affinity nucleotide analogue. Commonly used analogues include 2′-O-methyl-modified nucleic acids (2′-OMe) (RNA, 2006, 12, 163-176), 2′-O-(2-methoxyethyl)-modified nucleic acids (2′-MOE) (Nucleic Acids Research, 1998, 26, 16, 3694-3699), 2′-Deoxy-2′-fluoro-β-D-arabinoic acid (FANA) (Nucleic Acids Research, 2006, 34, 2, 451-461), Cyclohexene nucleic acids (CeNA) (Nucleic Acids Research, 2001, 29, 24, 4941-4947), Hexitol nucleic acids (HNA) and analogs hereof (Nucleic Acids Research, 2001, 29, 20, 4187-4194), Intercalating Nucleic Acids (INA) (Helvetica Chimica Acta, 2003, 86, 2090-2097) and 2′-O,4′-C-Ethylene-bridged-Nucleic Acids (ENA) (Bioorganic and Medicinal Chemistry Letters, 2002, 12, 1, 73-76). Additionally, in the present context, the oligonucleotide mimic referred to as peptide nucleic acid (PNA) (Nielsen et al., Science 254; 1497-1500, 1991 and U.S. Pat. Nos. 5,539,082; 5,714,331; and 5,719,262) is considered a high affinity nucleotide analogue. A preferred high affinity nucleotide analogue is LNA. A plurality of a combination of analogues may also be employed in an oligo of the invention.

As used herein, an oligo with an increased “binding affinity” for a recognition sequence compared to an oligo that includes the same sequence but does not include a nucleotide analog, refers to an oligo for which the association constant (Kₐ) of the recognition segment is higher than the association constant of the complementary strands of a double-stranded molecule. In a preferred embodiment, the association constant of the recognition segment is higher than the dissociation constant (K_{d}) of the complementary strand of the recognition sequence in the target sequence in a double stranded molecule.

Monomers are referred to as being “complementary” if they contain nucleobases that can form hydrogen bonds according to Watson-Crick base-pairing rules (e.g. G with C, A with T or A with U) or other hydrogen bonding motifs such as for example diaminopurine with T, 5-methyl C with G, 2-thiothymidine with A, inosine with C, pseudoisocytosine with G, etc.

As used herein, a probe with an increased “binding affinity” for a recognition sequence compared to a probe which comprises the same sequence but does not comprise a stabilizing nucleotide, refers to a probe for which the association constant (Kₐ) of the probe recognition segment is higher than the association constant of the complementary strands of a double-stranded molecule. In another preferred embodiment, the association constant of the probe recognition segment is higher than the dissociation constant (K_{d}) of the complementary strand of the recognition sequence in the target sequence in a double stranded molecule.

The terms “antagonising oligonucleotide” or “antagonising oligonucleotides” refer to oligonucleotides capable of hybridising to a target nucleic acid thereby counteracting the activity of the target nucleic acid.

Monomers are referred to as being “complementary” if they contain nucleobases that can form hydrogen bonds according to Watson-Crick base-pairing rules (e.g. G with C, A with T or A with U) or other hydrogen bonding motifs such as for example diaminopurine with T, 5-methyl C with G, 2-thiothymidine with A, inosine with C, pseudoisocytosine with G, etc.

The term “succeeding monomer” relates to the neighbouring monomer in the 5′-terminal direction and the “preceding monomer” relates to the neighbouring monomer in the 3′-terminal direction.

The term “target nucleic acid” or “target ribonucleic acid” refers to any relevant nucleic acid of a single specific sequence, e.g., a biological nucleic acid, e.g., derived from a patient, an animal (a human or non-human animal), a plant, a bacteria, a fungi, an archae, a cell, a tissue, an organism, etc. For example, where the target ribonucleic acid or nucleic acid is derived from a bacteria, archae, plant, non-human animal, cell, fungi, or non-human organism, the method optionally further comprises selecting the bacteria, archae, plant, non-human animal, cell, fungi, or non-human organism based upon detection of the target nucleic acid. In one embodiment, the target nucleic acid is derived from a patient, e.g., a human patient. In this embodiment, the invention optionally further includes selecting a treatment, diagnosing a disease, or diagnosing a genetic predisposition to a disease, based upon detection of the target nucleic acid.

“Target sequence” refers to a specific nucleic acid sequence within any target nucleic acid.

The terms “miRNA” or “microRNA” or “mature miRNA” refer to 17-25 nt, e.g., 21-25, non-coding RNAs derived from endogenous genes and in the present context also comprise the socalled mirtrons, produced from splicing of a short intron with hairpin potential (Berezikov et al., Mol. Cell. 28; 328-336, 2007). They are processed from longer (approximately 70 nucleotides in length) hairpin-like precursors termed pre-miRNAs. MiRNAs assemble in complexes termed miRNPs and recognize their targets by antisense complementarity. If the miRNAs match 100% of their target, i.e., the complementarity is complete, the target mRNA is cleaved, and the miRNA acts like a siRNA. If the match is incomplete, i.e., the complementarity is partial, then the translation of the target mRNA is blocked.

The term “off-target effect” refers to any instance in which the probe-analysis of a miRNA or other non-coding RNA causes an unintended effect by interacting either directly or indirectly with another miRNA sequence, a DNA sequence or a cellular protein or other moiety.

The term “false positive” refers to when a test result wrongly shows an effect or condition to be present. An off-target effect in the present context is considered a false positive.

The term “stringent conditions”, as used herein, is the “stringency” which occurs within a range from about Tₘ−5° C. (5° C. below the melting temperature (Tₘ) of the probe) to about 20° C. to 25° C. below Tₘ. As will be understood by those skilled in the art, the stringency of hybridization may be altered in order to identify or detect identical or related polynucleotide sequences. Hybridization techniques are generally described in Nucleic Acid Hybridization, A Practical Approach, Ed. Hames, B. D. and Higgins, S. J., IRL Press, 1985; Gall and Pardue, Proc. Natl. Acad. Sci., USA 63: 378-383, 1969; and John, et al. Nature 223: 582-587, 1969.

The term “intracellular avalibility” refers to intracellular probes, which are not entrapped in endosomes or other compartments and thereby free to hybridize with their targets found in cytoplasm and nucleus.

In the present context, the term “expression level” when refering to a nucleic acid or a translation product refers to the steady-state amount of the nucleic acid or translation product present as determined by methods known in the art and described herein.

In the present context, the term “phenotype” refers to any observed quality of a cell or organism, such as its morphology, development, or behaviou, its transcriptional or translational state, or other aspects of the biological state.

### EXAMPLES

The invention will now be further illustrated with reference to the following examples. It will be appreciated that what follows is by way of example only and that modifications to detail may be made while still falling within the scope of the invention.

### Materials and Methods:

TABLE 2
Oligonucleotide and sequences used in the experiments:

| | |
|---|---|
| Oligo name | Oligo sequence |

| | | |
|---|---|---|
| miR-24 3′ | 5′-GTTmCmCTGmCTG-3′ | (SEQ ID NO.: 7) |
| miR-24-5′ | 5′-AAmCTGAGmCmCA-3′ | (SEQ ID NO.: 8) |
| miR-24 22 mer LNA-DNA | 5′-cTgtTccTgcTgaActGagmCca-3′ | (SEQ ID NO.: 9) |
| miR-21 3′ | 5′-AAmCATmCAGT-3′ | (SEQ ID NO.: 5) |
| miR-21-5′ | 5′-TGATAAGmCTA-3′ | (SEQ ID NO.: 6) |
| miR-21 22 mer LNA-DNA | 5′-tmCaamCatmCagTctGatAagmCta-3′ | (SEQ ID NO.: 10) |
| Negative control (NC) | 5′-gTgtAacAcgTctAtamCgcmCca-3′ | (SEQ ID NO.: 11) |

### Synthesis of Oligonucleotides.

The oligonucleotide was synthesized on a 1-O-Dimethoxytrityl-propyl-disulflde,1′-succinyl-lcaa-CPG (Glenn Research, 20-2933-42) using standard procedure for LNA oligonucleotides (http://www.exiqon.com/uploads/lna_{—}11_-_lna_oligonucleotide_synthesis_-_an_overview(2).pdf). The oligonucleotides were deprotected using saturated aqueous ammonia at 55° C. for 6 h. and subsequently HPLC purified. Just prior to the conjugation with CPP the disulphide bridge was cleaved using DTT pH 8.5 for 16 h and subsequently desalted on a NAP-10 columns (GE Health Care, 17-0854-01). The conjugation with CPP was performed as described in the procedure provided by (Cambrex). The products were verified by MALDI-MS.

### Reporter Constructs.

The pMIR-21 and pMIR-24 reporter constructs were constructed by inserting a miR-21 or miR-24 complementary sequence in the 3′UTR of the pMIR-REPORT (Ambion) containing the firefly luciferase reporter gene. In short this was done by annealing oligonucleotide I (A: 5′-AAT GCA CTA GTT CAA CAT CAG TCT GAT AAG CTA GCT CAG CAA GCT TAA TGC-3′ (SEQ ID NO.: 12)) and II (B: 5′-GCA TTA AGC TTG CTG AGC TAG CTT ATC AGA CTG ATG TTG AAC TAG TGC ATT-3′ (SEQ ID NO.: 13)) comprising the miR-21 insert and for miR-24, (C: 5′-AAT GCA CTA GTC TGT TCC TGC TGA ACT GAG CCA GCT CAG CAA GCT TAA TGC-3′ (SEQ. ID NO.: 14)) and (D: 5′-GCA TTA AGC TTG CTG AGC TGG CTC AGT TCA GCA GGA ACA GAC TAG TGC ATT-3′ (SEQ ID NO.: 15)). Comprising the pMIR-24 insert. These fragments and the pMIR-REPORT vector were then digested with SpeI and HindIII and the fragments were subsequently cloned into the SpeI and HindIII sites of pMIR-REPORT vector using standard techniques, thereby generating pMIR-21 and pMIR-24. Cloning was confirmed by DNA sequencing through the miRNA insert sequence.

### Reporter Assays.

HeLa cells were propagated in Dulbecco's Modified Eagle's Minimal Essential Medium (DMEM) with Glutamax™ (Invitrogen) and supplemented with 10% foetal bovine serum (FBS). One day prior to transfection, cells were seeded in 96-well plates (Corning) at a density of 7000 cells/well. Cells were transfected using Xtreme Gene siRNA (Roche), with 70 ng/well of pMIR-21 reporter and 30 ng/well of the pGL4.73 Renilla (Promega) reporter plasmid for normalisation. Oligonucleotides were co-transfected with plasmid resulting in a final oligonucleotide concentration of 1-20 nM. After 3-4-h, media with transfection mix was removed and substituted with fresh media. Luciferase activities (Firefly and Renilla) were measured 24 h later using the Dual Glow Luciferase kit (Promega) on a BMG Optima luminometer.

After luminescence measurements relative light units (RLU) were corrected for background and firefly luminescence (FL) was normalised to Renilla luminescence (RL). Data presented in the diagram shows “fold up regulation” of the normalised (FL/RL) signal of the miRNA reporter vector relative to the no oligonucleotide control.

### Example 1

### Short LNA Oligonucleotides are Efficient miRNA Inhibitors

To measure the effect of miRNA antagonising oligonucleotides a luciferase based miR-21 and miR-24 sensor reporter were constructed. These reporters harbour a sequence fully complementary to hsa-miR-21 and hsa-miR-24 respectively. When the reporter mRNA is recognized by a miR-21 or miR-24 containing RISC complex, the luciferase encoding mRNA is cleaved and subsequently degraded. The luciferase expression levels thereby reflect the endogenous level of active miR-21 and miR-24.

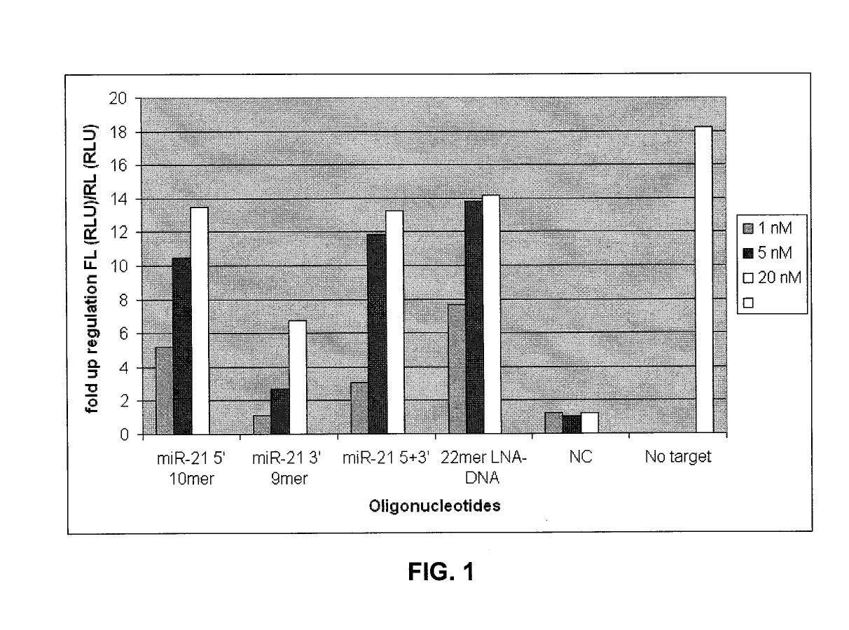

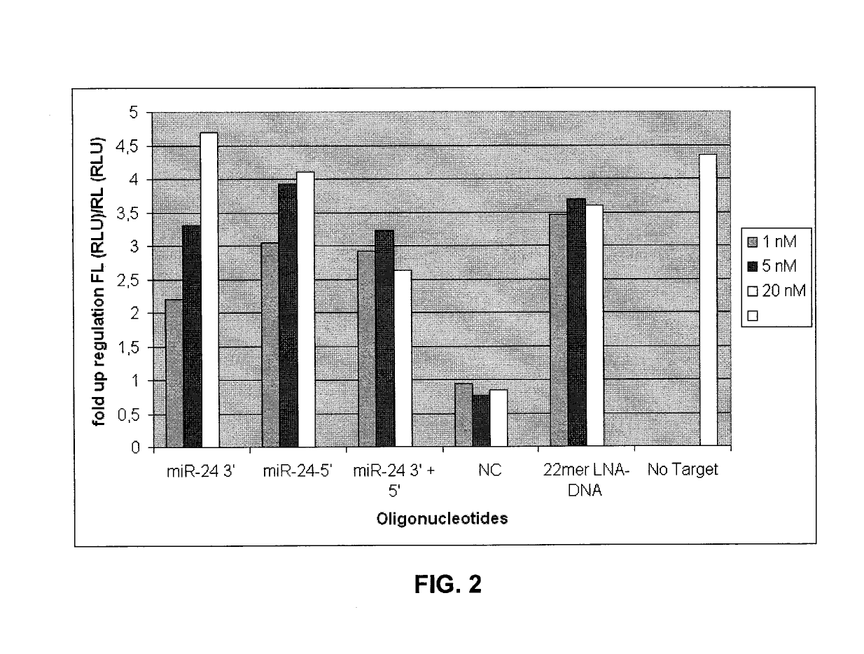

A wide variety of cell lines are known to express miR-21 and miR-24 at high levels. In one line of experiments reporter plasmids, pMIR-21 and pMIR-24, and miR-21 and miR-24 inhibiting oligonucleotides were co-transfected (see materials and methods). Reporter data show that when co-transfected with plasmid all oligonucleotides showed efficient knock down of their target miRNA sequence as inhibition of endogenous miR-21 resulted in a 5-15 fold increase in expression of the miR-21 sensor reporter (FIG. 1). For the miR-24 reporter (FIG. 2) knockdown was also very efficient resulting in complete relief of miR repression, as the miR-24 reporter constructs is up regulated to the same expression level as the parental vector with no miR-24 target sequence (No target).

For both miR-24 and miR-21 there seemed to be no synergistic effect of co-transfecting both 5′ end and 3′end targeting short oligos.

These data proves the concept that two non-overlapping oligos can be directed to target the same miRNA and thereby enables reagent redundancy experiments in order to reduce the risk of reporting false phenotypes due to sequence specific off-target effects.

1. A method of determining whether a phenotype induced by an antagonizing oligonucleotide for a target non-coding RNA is a false positive, said method comprising:
(a) introducing a first antagonizing oligonucleotide into a first target cell, wherein said first antagonizing oligonucleotide comprises a recognition sequence hybridizing to a first region of said target RNA;
(b) measuring a phenotype in said first target cell after (a);
(c) introducing a second antagonizing oligonucleotide into a second target cell, wherein said second antagonizing oligonucleotide comprises a recognition sequence hybridizing to a second region of said target RNA;
(d) measuring a phenotype in said second target cell after (c); and
(e) comparing the phenotype in said first target cell with the phenotype in said second target cell,
wherein, if the phenotype in said first target cell is similar to the phenotype in said second target cell, the phenotype observed in said first target cell is a false positive.

2.-17. (canceled)

18. The method according to claim 1, wherein said first antagonizing oligonucleotide comprises at least one high affinity nucleic acid analog.

19. The method according to claim 1, wherein said second antagonizing oligonucleotide comprises at least one high affinity nucleic acid analog.

20. The method according to claim 1, wherein both said first antagonizing oligonucleotide and said second antagonizing oligonucleotide comprise at least one high affinity nucleic acid analog.

21. The method according to claim 20, wherein the at least one high affinity nucleic acid analog is a Locked Nucleic Acid (LNA).

22. The method according to claim 21, wherein said non-coding RNA is a miRNA.

23. The method according to claim 22, wherein said miRNA is a mature miRNA.

24. The method according to claim 22 or 23, wherein said first region of said target RNA comprises the 3′ end of said target and said second region of said targetz RNA comprises the 5′ end of said target.

25. The method according to claim 24, wherein said first antagonizing oligonucleotide and said second antagonizing oligonucleotide comprise from 5 to 15 monomer subunits.

26. The method according to claim 25, wherein said first antagonizing oligonucleotide and said second antagonizing oligonucleotide comprise from 8 to 13 monomer subunits.

27. Use of at least two antagonizing oligonucleotides for a target non-coding RNA, such as a mature miRNA, for determining whether a phenotype induced by an antagonizing oligonucleotide for the target non-coding RNA is a false positive.

28. A kit comprising at least one antagonizing oligonucleotide hybridizing to a first region of a target non-coding RNA and a second antagonizing oligonucleotide hybridizing to a second region of the target non-coding RNA.

29. The kit according to claim 28, wherein said target non-coding RNA is a miRNA.

30. The kit according to claim 29, wherein said miRNA is a mature miRNA.

31. The kit according to claim 29 or 30, wherein both said first antagonizing oligonucleotide and said second antagonizing oligonucleotide comprise at least one high affinity nucleic acid analog.

32. The kit according to claim 31, wherein the at least one high affinity nucleic acid analog is a Locked Nucleic Acid (LNA).

33. The kit according to claim 32, wherein said first region of said target non-coding RNA comprises the 3′ end of said target and said second region of said target non-coding RNA comprises the 5′ end of said target.

34. The kit according to claim 33, wherein said first antagonizing oligonucleotide and said second antagonizing oligonucleotide comprise from 5 to 15 monomer subunits.

35. The kit according to claim 34, wherein said first antagonizing oligonucleotide and said second antagonizing oligonucleotide comprise from 8 to 13 monomer subunits.


SEQUENCE LISTING (11 sequences)

SEQ ID NO: 1 - RNA, 22 nt, Homo sapiens
uagcuuaucagacugauguuga

SEQ ID NO: 2 - DNA, 22 bp, Artificial Sequence
  Synthetic
atcgaatagtctgactacaact

SEQ ID NO: 3 - DNA, 10 bp, Artificial Sequence
  Synthetic sequence
atcgaatagt

SEQ ID NO: 4 - DNA, 9 bp, Artificial Sequence
  Synthetic
tgactacaa

SEQ ID NO: 5 - DNA, 9 bp, Artificial Sequence
  Synthetic
aacatcagt

SEQ ID NO: 6 - DNA, 10 bp, Artificial Sequence
  Synthetic
tgataagcta

SEQ ID NO: 7 - DNA, 10 bp, Artificial Sequence
  Synthetic
gttcctgctg

SEQ ID NO: 8 - DNA, 10 bp, Artificial Sequence
  Synthetic
aactgagcca

SEQ ID NO: 9 - DNA, 22 bp, Artificial Sequence
  Synthetic
ctgttcctgctgaactgagcca

SEQ ID NO: 10 - DNA, 22 bp, Artificial Sequence
  Synthetic
tcaacatcagtctgataagcta

SEQ ID NO: 11 - DNA, 22 bp, Artificial Sequence
  Synthetic
gtgtaacacgtctatacgccca